(19) United States
(12) Patent Application Publication
(10) Pub. No.: US 2003/0015205 A1
(43) Pub. Date: Jan. 23, 2003

(54) Face mask

(76) Inventor: Fang-Hoang Lai, Taipei (TW)

Correspondence Address: BROWDY AND NEIMARK, P.L.L.C., 624 NINTH STREET, NW, SUITE 300, WASHINGTON, DC, 20001-5303, US

(21) Appl. No.: 09/907,699

(22) Filed: Jul. 19, 2001

(51) Int. Cl. A61F 11/00

(52) U.S. Cl. 128/857

(57) ABSTRACT

A face mask comprises a water-containing piece layer for covering most area of a human face to moisten the face skin. The piece layer is provided with a hollowed portion corresponding in location to nostril, and in the inner side with a ventilation structure enabling the face skin to breathe.

### FIELD OF THE INVENTION

The present invention relates generally to a face mask, and more particularly to a face mask to keep the face skin moisture.

### BACKGROUND OF THE INVENTION

The face skin of an airplane passenger is vulnerable to loss of moisture in view of the change in pressure and moisture of the airplane cabin in relation to the ground. The vulnerability is especially acute in a long trip, thereby resulting in an excessive loss of moisture in the face skin of the traveler. Similar situation may also take place in train, bus, theater, sauna, office, home, etc. It is well known that the excessive loss of skin moisture can result in a bodily discomfort as well as a poor facial appearance, and that the excessive loss of the facial skin moisture can adversely affect the cosmetic preparation of the face.

### SUMMARY OF THE INVENTION

The primary objective of the present invention is to provide a face mask to minimize the loss of facial skin moisture.

The face mask of the present invention comprises a water-containing piece layer which is sized to cover most of the human face and is provided with a breathable portion corresponding in location to the human nose. The water containing piece layer is provided with a ventilation structure enabling the facial skin to breathe.

### BRIEF DESCRIPTION OF THE DRAWINGS

FIG. 1 shows a front view of a first preferred embodiment of the present invention.

FIG. 2 shows a rear view of the first preferred embodiment of the present invention.

FIG. 3 shows a sectional schematic view of the first preferred embodiment of the present invention.

FIG. 4 shows a schematic view of the first preferred embodiment of the present invention in use.

FIG. 5 shows a front view of a second preferred embodiment of the present invention.

FIG. 6 shows a rear view of the second preferred embodiment of the present invention.

FIG. 7 shows a sectional schematic view of the second preferred embodiment of the present invention.

FIG. 8 shows an external schematic view of another embodiment pattern of the present invention.

FIGS. 9 and 10 are sectional schematic views of another two embodiment patterns of the present invention.

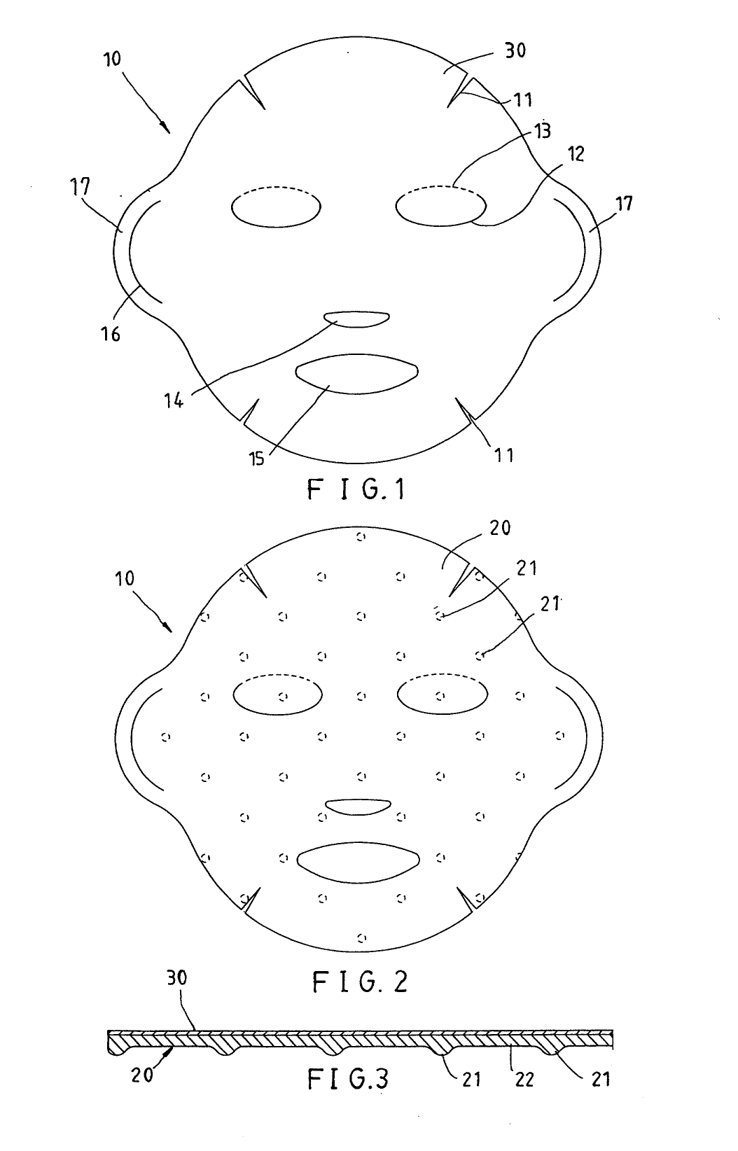

### DETAILED DESCRIPTION OF THE INVENTION

As shown in the drawings provided, a face mask 10 has a main body which is a water-containing piece layer 20 made of a water absorbable fiber material, such as fabric, no-woven fabric, cotton cloth, paper, etc. The layer is provided in the inner side with a knurled surface which is formed of a plurality of protuberances 21 and depressions 22 intermingled with the protuberances 21. The protuberances 21 are round in shaped and are regularly distributed such that they are separated by a distance ranging between 20 mm and 25 mm. The protuberances 21 may have other shapes and may be arranged irregularly. The layer 20 is provided in the surface with a plastic film layer 30 having a waterproof effect. The face mask 10 has a profile corresponding to the shape of the human face. The face mask 10 is worn on the human face such that one side of the film layer 30 faces outward. The face mask 10 is provided in the periphery with a plurality of expansion seams 11 extending inward from the outer edge and enabling the face mask 10 to fit the human face profile. The face mask 10 is provided with two arcuate open seams 12 corresponding in location to the human eyes to facilitate the exposing of the human eyes. The face mask 10 is provided with two dotted line seams 13, which are symmetrical with the open seams 12 respectively, to facilitate the forming of two oval openings at the position of eyes in conjunction with the open seams 12. The face mask 10 is provided with a nose opening 14 and a mouth opening 15. The face mask 10 is provided in left and right sides with a longitudinal split seam 16 corresponding in location to ear. The split seam 16 is provided in the outer side with an annular ear ribbon 17 expandable outwards.

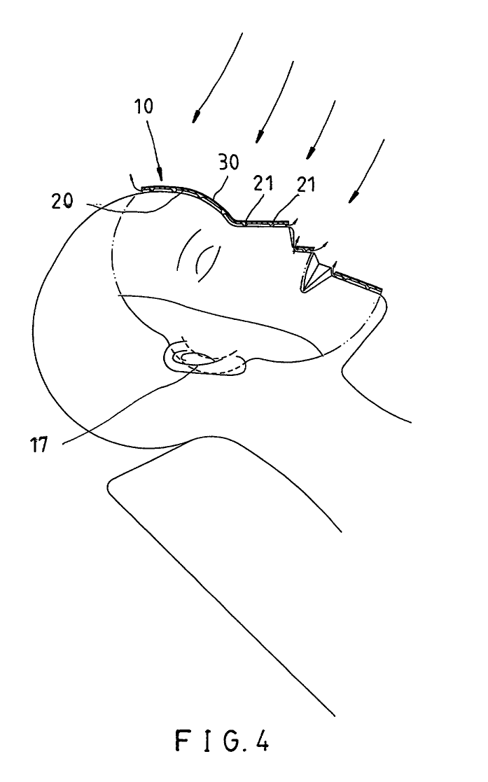

In production, the layer 20 is first provided with a purified water, a cosmetic water, or an aromatic liquid, and is then placed in a waterproof and airtight bag. In use, the face mask 10 is removed from the bag to wear on the face such that the water-containing piece layer 20 faces inward, and that the film layer 30 faces outward, as shown in FIG. 4. The piece layer 20 comes in contact with the face skin. The two ear ribbons 17 are fitted over the ears. In light of the nose opening 13 and the mouth opening 14, the breathing of the wearer is not obstructed. In addition, the face mask 10 does not prevent its wearer from speaking or drinking. In light of the open seam 12 and the dotted line seam 13, the face mask of the present invention does not obstruct the eye sight of the wearer. The plastic film layer 30 is capable of separating the water and preventing the water of the layer 20 from evaporating rapidly, especially in a dry cool environment, thereby prolonging the effect of moistening the face skin. The protuberances 21 of the piece layer 20 come in contact with the face skin gently. As a result, the knurled surface of the inner side of the piece layer 20 forms a ventilation structure to promote the air circulation between the piece layer 20 and the face skin, thereby bringing about a soothing effect on the face.

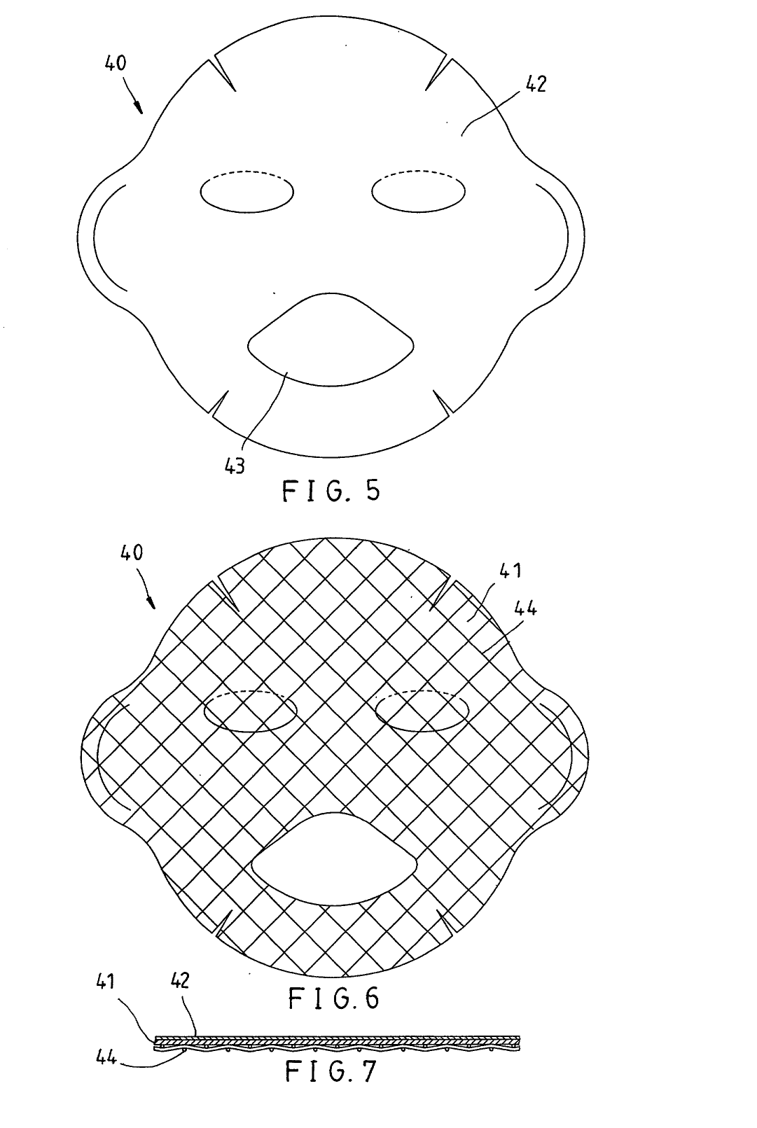

As shown in FIGS. 5 and 6, a face mask 40 of the second preferred embodiment has a main body which is a water-containing piece layer 41 which is provided with a waterproof plastic film layer 42 and a mouth-nose opening 43 corresponding in location to nostril and jaw. In addition, the piece layer 41 is provided with a meshed layer 44 of cotton, which comes in contact with the face skin to bring about a ventilation effect. In other words, the meshed layer 44 serves to soothe the face skin.

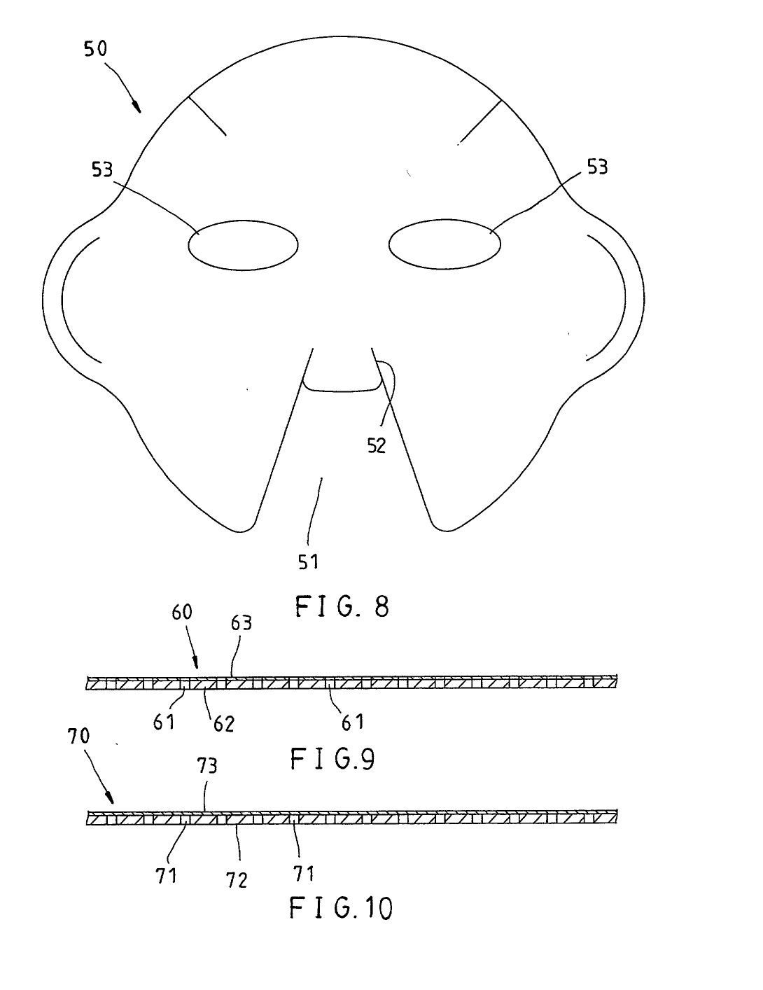

As shown in FIG. 8, the face mask 50 is designed to mask the face partially and is provided with a hollow portion 51 which is corresponding to the bottom end of the face such that the hollow portion 51 extends upwards from the chin to the nose. An expansion seam 52 is provided such that it extends upward along each of two nose wings. The present invention is provided with two oval eye openings 53 corresponding in location to the eyes.

As shown in FIG. 9, the face mask 60 is provided with a plurality of ventilation holes 61 which extend from the inner side of the water-containing piece layer 62 to the surface of the plastic film layer 63. The ventilation holes 61 reduces the contact area between the piece layer 62 and the face skin. In the meantime, the ventilation holes 61 promote the breathing of the face skin. As shown in FIG. 10, the plastic film layer 73 is breathable and capable of stopping water. The ventilation holes 71 extends through only the water-containing piece layer 72 without compromising the ventilation effect.

The plastic film layer of the present invention is colored or knurled. The present invention may be devoid of the plastic film layer which is intended to prolong the use of the face mask.

The face mask of the present invention is suitable for use in all transportation means and environments, especially in the cool dry environment. In addition, the face mask of the present invention is cost-effective and is an ideal adjunct for travelers.

What is claimed is:

1. A face mask comprising a water containing piece layer for covering a person's face to moisten the face skin, said piece layer having a hollowed portion corresponding in location to nostril, said piece layer provided in an inner side with a ventilation structure to promote air circulation between said piece layer and the face skin.

2. The face mask as defined in claim 1, wherein said water containing piece layer is provided with a plastic film layer attached thereto.

3. The face mask as defined in claim 1, wherein said water-containing piece layer is provided in the inner side with a knurled surface formed of a plurality of protuberances and depressions for forming said ventilation structure.

4. The face mask as defined in claim 3, wherein said protuberances are round in shape and are regularly arranged such that said protuberances are separated from one another by a distance ranging between 20 mm and 25 mm.

5. The face mask as defined in claim 1, wherein said water-containing piece layer is provided in the inner side with a meshed layer attached thereto for forming said ventilation structure.

6. The face mask as defined in claim 1, wherein said water-containing piece layer is provided with a plurality of ventilation holes extending from the inner side thereof to the surface thereof for reducing contact area between said piece layer and the face skin, said ventilation holes forming said ventilation structure.

7. The face mask as defined in claim 2, wherein said plastic film layer is permeable to air; wherein said water containing piece layer is provided with a plurality of ventilation holes extending from the inner side thereof to the surface thereof to form said ventilation structure.

8. The face mask as defined in claim 2, wherein said face mask is provided with a plurality of ventilation holes extending from the inner side of said water-containing piece layer to the surface of said plastic film layer to form said ventilation structure.

9. The face mask as defined in claim 1, wherein said face mask is provided with a nose opening corresponding in location to nostril, and a mouth opening corresponding in location to mouth.

10. The face mask as defined in claim 1, wherein said face mask is provided with a mouth-nose opening corresponding in location to nostril and mouth.

11. The face mask as defined in claim 1, wherein said face mask is provided with a hollowed portion corresponding to a portion extending from chin to nostril.

12. The face mask as defined in claim 1, wherein said face mask is provided with two open seams corresponding to two eyes for exposing the eyes by lifting upper portions of said open seams.

13. The face mask as defined in claim 12, wherein said open seams are arcuate in shape and said face mask is further provided with a symmetrical dotted line seam forming an oval shape along with said open seam.

14. The face mask as defined in claim 1, wherein said face mask is provided with two eye openings corresponding in location to two eyes.

15. The face mask as defined in claim 1, wherein said face mask is provided in left side and right side with a longitudinal split seam which is provided in outer side with an annular ear ribbon expandable outwards.

16. The face mask as defined in claim 1, wherein said face mask is provided in the periphery with a plurality of expansion seams extending inwards from the outer edge.
